(19) United States
(12) Patent Application Publication
(10) Pub. No.: US 2025/0143979 A1
(43) Pub. Date: May 8, 2025

(54) WATER-IN-OIL TYPE SOLID STICK COMPOSITION CONTAINING A LARGE AMOUNT OF WATER PHASE

(71) Applicant: AMOREPACIFIC CORPORATION, Seoul (KR)

(72) Inventors: Shinbeom KANG, Yongin-si (KR); Byungryol PAIK, Yongin-si (KR); Byungfhy SUH, Yongin-si (KR); Jongsuk LEE, Yongin-si (KR)

(73) Assignee: AMOREPACIFIC CORPORATION, Seoul (KR)

(21) Appl. No.: 18/933,337

(22) Filed: Oct. 31, 2024

(30) Foreign Application Priority Data
Nov. 2, 2023 (KR) 10-2023-0149839

(51) Int. Cl. A61K 8/02; A61K 8/19; A61K 8/368; A61K 8/37; A61K 8/49; A61K 8/58

(52) U.S. Cl. CPC A61K 8/0229; A61K 8/19; A61K 8/368; A61K 8/37; A61K 8/498; A61K 8/585

(57) ABSTRACT

The present disclosure discloses a water-in-oil type solid stick composition containing a large amount of water phase that can obtain a stable formulation while containing an acidic active ingredient, and can have a water-bursting effect immediately upon applying to the skin by containing a large amount of water phase. According to the present disclosure, the solid stick formulation can be stabilized without pH restriction, and is also excellent in terms of safety because PEG and silicone oil are not used.

### CROSS REFERENCE TO RELATED APPLICATION

The present application claims the priority of Korean Patent Application No. 10-2023-0149839, filed on Nov. 2, 2023, the entire contents of which is incorporated herein for all purposes by this reference.

### FIELD OF THE INVENTION

The present specification discloses a solid stick composition containing a large amount of water phase, the composition comprising an acidic ingredient and having a stable formulation.

### BACKGROUND

Existing stick-type cosmetic compositions are classified into a SOAP (containing a fatty acid and a neutralizer) emulsified formulation and an oil-type (anhydrous) product.

The SOAP (containing the fatty acid and the neutralizer) emulsified formulation is divided into a W/O formulation and an O/W formulation, which are implemented at pH 8 or higher due to the technical characteristics thereof, and have a risk of stability.

Further, since the W/O formulation contains a PEG-based surfactant or a silicone-based oil without containing a large amount of moisture moisturizing ingredients, and the O/W stick product contains a PEG-based or silicone-based oil, they have poor compatibility with a makeup product (W/O) and have the problem that the effective ingredients are easily washed away by water or sweat.

Furthermore, the oil (anhydrous) formulation contains an excessive amount of powder due to excessive oiliness, and therefore it was pointed out as a disadvantage that there is no moisture feeling when used.

In particular, if the above products contain acidic ingredients, they have formulation stability issues, which makes it difficult to contain acidic active ingredients such as vitamin C.

Accordingly, there is a need to develop a composition that has formulation stability, can contain various ingredients while being stable, and has excellent moisture feeling by complementing the shortcomings of the composition in the stick formulation as described above.

### SUMMARY OF THE INVENTION

In an aspect of the present disclosure, it is an object to provide a water-in-oil type solid stick composition containing a large amount of water phase that contains an acidic ingredient, does not contain a PEG-based surfactant and a silicone oil, and has formulation stability.

In an aspect, the present disclosure provides a water-in-oil type solid stick composition containing a large amount of water phase, the solid stick composition comprising an oil phase and a water phase, wherein the oil phase contains a silicone emulsifier containing an ethoxy glucoside derivative, the water phase contains an acidic ingredient, and the water phase is 35 wt % or more based on the total weight of the solid stick composition.

In an aspect, a solid stick composition disclosed in the present specification can obtain a stable formulation while containing an acidic active ingredient, including a specific silicone elastomer, and can have a water-bursting effect immediately upon applying to the skin by containing a large amount of water phase.

### DETAILED DESCRIPTION OF THE INVENTION

Hereinafter, the present disclosure will be described through the following Examples in more detail. However, the following Examples are provided merely for the purpose of illustration to help understanding of the present disclosure, and the scope and range of the present disclosure are not limited thereto.

In exemplary embodiments of the present disclosure, there is provided a water-in-oil type solid stick composition containing a large amount of water phase, the solid stick composition comprising an oil phase and a water phase, wherein the oil phase contains a silicone emulsifier containing an ethoxy glucoside derivative, the water phase contains an acidic ingredient, and the water phase is 35 wt % or more based on the total weight of the solid stick composition.

In an embodiment, the composition may be a skin moisturizing composition.

In an embodiment, the composition may be a skin whitening composition.

In an embodiment, the composition may be a skin anti-aging composition.

In another exemplary embodiment of the present disclosure, there is provided a use of a water-in-oil type solid stick composition containing a large amount of water phase for preparing the skin moisturizing composition, the solid stick composition comprising an oil phase and a water phase, wherein the oil phase contains a silicone emulsifier containing an ethoxy glucoside derivative, the water phase contains an acidic ingredient, and the water phase is 35 wt % or more based on the total weight of the solid stick composition.

In another exemplary embodiment of the present disclosure, there is provided a use of a water-in-oil type solid stick composition containing a large amount of water phase for preparing the skin whitening composition, the solid stick composition comprising an oil phase and a water phase, wherein the oil phase contains a silicone emulsifier containing an ethoxy glucoside derivative, the water phase contains an acidic ingredient, and the water phase is 35 wt % or more based on the total weight of the solid stick composition.

In another exemplary embodiment of the present disclosure, there is provided a use of a water-in-oil type solid stick composition containing a large amount of water phase for preparing the skin anti-aging composition, the solid stick composition comprising an oil phase and a water phase, wherein the oil phase contains a silicone emulsifier containing an ethoxy glucoside derivative, the water phase contains an acidic ingredient, and the water phase is 35 wt % or more based on the total weight of the solid stick composition.

In another exemplary embodiment of the present disclosure, there is provided a method for improving skin moisturizing power, comprising applying an effective amount of the aforementioned water-in-oil type solid stick composition containing a large amount of water phase to the skin of a subject in need thereof.

In another exemplary embodiment of the present disclosure, there is provided a method for improving skin whitening effect, comprising applying an effective amount of the aforementioned water-in-oil type solid stick composition containing a large amount of water phase to the skin of a subject in need thereof.

In another exemplary embodiment of the present disclosure, there is provided a method for improving skin anti-aging power, comprising applying an effective amount of the aforementioned water-in-oil type solid stick composition containing a large amount of water phase to the skin of a subject in need thereof.

The composition according to an embodiment of the present disclosure may stabilize a formulation while containing a high content of an effective water-soluble ingredient. In addition, the composition may provide a moisture feeling like water bursting immediately after application to allow a consumer to feel moisturizing and moisture at the same time.

For example, the water phase may be 35 wt % or more, 40 wt % or more, 45 wt % or more, 50 wt % or more, 55 wt % or more, 60 wt % or more, 65 wt % or more, 70 wt % or more, 75 wt % or more, or 80 wt % or more, based on the total weight of the solid stick composition and may be 90 wt % or less, 85 wt % or less, 80 wt % or less, 75 wt % or less, 70 wt % or less, 65 wt % or less, 60 wt % or less, or 55 wt % or less, based on the total weight of the solid stick composition.

In an embodiment, a pH of the composition may be 3 to 6.

For example, the pH may be 3 or more, 3.5 or more, 4 or more, 4.5 or more, 5 or more, or 5.5 or more, and may be 6 or less, 5.5 or less, 5 or less, 4.5 or less, 4 or less, or 3.5 or less.

The water-in-oil type solid stick composition containing a large amount of water phase according to the present disclosure may comprise various ingredients without pH restriction, and the formulation can be stabilized by adjusting the pH of the composition within the above range.

In an embodiment, the silicone emulsifier containing the ethoxy glucoside derivative may be present in an amount of 0.001 to 10 wt % based on the total weight of the composition.

For example, the silicone emulsifier containing the ethoxy glucoside derivative may be 0.001 wt % or more, 0.01 wt % or more, 0.1 wt % or more, 0.5 wt % or more, 1 wt % or more, 2 wt % or more, 3 wt % or more, 4 wt % or more, 5 wt % or more, 6 wt % or more, 7 wt % or more, 8 wt % or more, or 9 wt % or more, based on the total weight of the composition, and may be 10 wt % or less, 9 wt % or less, 8 wt % or less, 7 wt % or less, 6 wt % or less, 5 wt % or less, 4 wt % or less, 3 wt % or less, 2 wt % or less, 1 wt % or less, 0.5 wt % or less, 0.1 wt % or less, or 0.01 wt % or less, based on the total weight of the composition.

In an embodiment, the silicone emulsifier containing the ethoxy glucoside derivative may include at least one selected from caprylyl dimethicone ethoxy glucoside and dimethicone ethoxy glucoside.

In an embodiment, the oil phase may further include at least one selected from the group consisting of hydrocarbon oil, a wax, and an additional emulsifier.

In an embodiment, the carbon number of the hydrocarbon oil may be 20 or less.

According to the present disclosure, the solid stick formulation can have a refreshing and light feeling of use even if the hydrocarbon oil having a carbon number of 20 or less is used.

For example, the carbon number of the hydrocarbon oil may be 20 or less, 19 or less, 18 or less, 17 or less, 16 or less, or 15 or less.

In an embodiment, the hydrocarbon oil may include at least one selected from the group consisting of isododecane and dicaprylyl carbonate.

In an embodiment, the hydrocarbon oil may be present in an amount of 5 to 12 wt % based on the total weight of the solid stick composition.

For example, the content of the hydrocarbon oil may be 5 wt % or more, 7 wt % or more, 9 wt % or more, or 11 wt % or more, and may be 12 wt % or less, 10 wt % or less, 8 wt % or less, or 6 wt % or less.

In an embodiment, a melting point of the wax may be 50° C. or more.

For example, the melting point of the wax may be 100° C. or lower. If the melting point is lower than 50° C., a hardness of the composition may decrease to collapse the solid stick formulation.

For example, the wax may include at least one selected from the group consisting of a synthetic wax, OZOKERITE, and a microcrystalline wax.

In an embodiment, the wax may be present an amount of 5 to 10 wt % based on the total weight of the solid stick composition.

For example, the wax may be 5 wt % or more, 7 wt % or more, or 9 wt % or more, based on the total weight of the solid stick composition, and may be 10 wt % or less, 8 wt % or less, or 6 wt % or less, based on the total weight of the solid stick composition.

In an embodiment, the additional emulsifier may include at least one of a silicone-based emulsifier and a polyglyceryl-based non-silicone-based emulsifier.

For example, the silicone-based emulsifier included in the additional emulsifier may be a silicone-based emulsifier containing polyglyceryl.

In an embodiment, the silicone-based emulsifier may include at least one of lauryl polyglyceryl-3-polydimethylsiloxyethyl dimethicone and polyglyceryl-3-polydimethylsiloxyethyl dimethicone.

In an embodiment, the polyglyceryl-based non-silicone-based emulsifier may include at least one selected from the group consisting of polyglyceryl-5-polyricinoleate, polyglyceryl-4-isostearate, polyglyceryl-3-polyricinoleate, polyglyceryl-2-dipolyhydroxystearate, diisostearoyl polyglyceryl-3-dimer dilinoleate, polyglyceryl-4-diisostearate/polyhydroxystearate/sebacate, polyglyceryl-2-isostearate, polyglyceryl-3-diisostearate, polyglyceryl-2-isostearate/dimer dilinoleate copolymer.

In an embodiment, the additional emulsifier may be present in an amount of 0.1 to 5 wt % based on the total weight of the solid stick composition.

For example, the additional emulsifier may be 0.1 wt % or more, 0.5 wt % or more, 1 wt % or more, 2 wt % or more, 3 wt % or more, or 4 wt % or more, based on the total weight of the solid stick composition, and may be 5 wt % or less, 4 wt % or less, 3 wt % or less, 2 wt % or less, 1 wt % or less, or 0.5 wt % or less, based on the total weight of the solid stick composition.

For example, the acidic ingredient is an ingredient that releases H⁺ in an aqueous solution, and mainly consists of an organic acid having a carbon atom in the molecule, but is not limited thereto.

In an embodiment, the acidic ingredient may be an organic acid.

In an embodiment, the acidic ingredient may include at least one selected from the group consisting of a polyhydroxy acid (PHA), a beta hydroxy acid (BHA), an alpha hydroxy acid (AHA), a pure vitamin C, and a vitamin C derivative.

According to the present disclosure, the solid stick composition makes possible it to contain various acidic ingredients that are effective water-soluble ingredients without pH restriction, and in particular, to expect an effect of allowing an external phase (oil phase) to block unstable materials such as vitamin C that are vulnerable to oxygen and heat of the outside, thereby stabilizing the effective ingredients.

In an embodiment, the acidic ingredient may include at least one selected from the group consisting of gluconolactone, salicylic acid, glycolic acid, lactic acid, citric acid, ascorbic acid, ethyl ascorbic acid, succinic acid, and ferulic acid.

In an embodiment, the acidic ingredient may be present in an amount of 1 to 15 wt % based on the total weight of the solid stick composition.

In an embodiment, the composition may not comprise a PEG-based surfactant and silicone oil.

In an embodiment, the composition may be a cosmetic composition.

The cosmetic composition according to the present disclosure may additionally comprise a functional additive and ingredients contained in a general cosmetic composition. The functional additive may include ingredients selected from the group consisting of a water-soluble vitamin, an oil-soluble vitamin, a high molecular peptide, a high molecular polysaccharide, squalane, a sphingolipid, and a seaweed extract.

The cosmetic composition according to the present disclosure may be also mixed with ingredients contained in a general cosmetic composition, if necessary, in addition to the functional additive. Further, the mixed ingredients may include an oil and fat ingredient, a moisturizer, an emollient, an emulsifier, organic and inorganic pigments, organic powders, a ultraviolet absorber, a preservative, a sterilizer, an antioxidant, a plant extract, a pH adjuster, alcohol, a pigment, a fragrance, a blood circulation promoter, a cold sensing agent, an antiperspirant, purified water, etc.

In an embodiment, the composition may be a composition for external application in the skin.

The above skin external application agent is a general term that can include anything applied externally to the skin, and may include various types of cosmetics.

Hereinafter, the present disclosure will be described through the following Examples in more detail. However, the following Examples are provided merely for the purpose of illustration to help understanding of the present disclosure, and the scope and range of the present disclosure are not limited thereto.

### Example—Preparation of W/O Solid Stick Containing a Large Amount of Water Phase

According to Table 1 below, solid stick compositions were prepared with various contents (unit: weight %). Specifically, they were prepared according to the following A-B-C steps.

Step A: [Oil, wax, silicone emulsifier containing ethoxy glucoside derivative, silicone emulsifier] part: Dissolving by heating and stirring at 80° C. or higher.

Step B: [Acidic ingredient, water phase (35˜85%)] part: Dissolving by heating and stirring at 80° C. or higher.

Step C: Adding a mixture prepared by step B to a mixture prepared by step A and stirring them.

In the following Table 1, Examples 1-3 of the present disclosure employed a silicone emulsifier containing an ethoxy glucoside derivative as a silicone elastomer.

TABLE 1

| | Comparative | Comparative | Comparative | | | |
|---|---|---|---|---|---|---|
| | Example 1 | Example 2 | Example 3 | Example 1 | Example 2 | Example 3 |
| Acidic | PHA | PHA | PHA | PHA | PHA | PHA |
| ingredient | (gluconolactone | (gluconolactone | (gluconolactone | (gluconolactone | (gluconolactone | (gluconolactone |
| | 10%) | 10%) | 10%) | 10%) | 10%) | 10%) |
| | BHA | BHA | BHA | BHA | BHA | BHA |
| | (SALICYCLIC | (SALICYCLIC | (SALICYCLIC | (SALICYCLIC | (SALICYCLIC | (SALICYCLIC |
| | ACID 2%) | ACID 2%) | ACID 2%) | ACID 2%) | ACID 2%) | ACID 2%) |
| * Water | 85% | 85% | 85% | 85% | 85% | 85% |
| phase | | | | | | |
| Oil | Polybutene | Dicaprylyl | Dicaprylyl | Dicaprylyl | Isododecane | Dicaprylyl |
| (non- | 5% | carbonate 5% | carbonate 5% | carbonate | 5% | carbonate |
| silicone- | | | | 5% | | 12% |
| based | | | | | | |
| oil) | | | | | | |
| Wax | Synthetic wax | Synthetic wax | Synthetic | Synthetic | Synthetic | Synthetic |
| | 7% (MP 50° C. | 7% (MP less | wax 7% (MP | wax 7% | wax 7% | wax 7% |
| | or higher) | than 50° C.) | 50° C. or | (MP 50° C. | (MP 50° C. | (MP 50° C. |
| | | | higher) | or higher) | or higher) | or higher) |
| Silicon | [Caprylyl | | [Caprylyl | [Caprylyl | [Caprylyl | [Caprylyl |
| emulsifier | dimethicone | | dimethicone | dimethicone | dimethicone | dimethicone |
| containing | ethoxy | | ethoxy | ethoxy | ethoxy | ethoxy |
| ethoxy | glucoside] | | glucoside] | glucoside] | glucoside] | glucoside] |
| glucoside | 0.015% | | 0.515% | 0.015% | 0.15% | 10.015% |
| derivative | | | | | | |
| Silicone | [Lauryl | [Lauryl | Not | [Lauryl | [Lauryl | [polyglyceryl- |
| emulsifier | polyglyceryl- | polyglyceryl- | containing | polyglycer | polyglycer | 3- |
| | 3- | 3- | | yl-3- | yl-3- | polydimethyl |
| | polydimethyl- | polydimethyl- | | polydimethyl | polydimethyl | siloxyethyl |
| | siloxyethyl | siloxyethyl | | siloxyethyl | siloxyethyl | dimethicone]: |
| | dimethicone]: | dimethicone]: | | dimethicone]: | dimethicone]: | HLB = 3~4 |
| | HLB = 3~4 | HLB = 3~4 | | HLB = 3~4 | HLB = 3~4 | (0.5%) |
| | (0.5%) | (0.515%) | | (0.5%) | (0.5%) | |
| Observation | Heavy feeling | Poor | Poor | O (well- | Excellent | O (well- |
| result | of use | emulsification | emulsification | made | formulation | made |
| | | | | formulation) | stability, | formulation) |
| | | | | Excellent | but | Excellent |
| | | | | formulation | clammy | formulation |
| | | | | stability, | feeling of | stability, |
| | | | | excellent | use and | excellent |
| | | | | water- | slightly | water- |
| | | | | bursting | reduced | bursting |
| | | | | feel | water- | feel |
| | | | | | bursting | |
| | | | | | feel | |

* Water phase (including acidic ingredients, 85 wt %) = Purified water (up to 100), Sodium chloride (1%), GLUCONOLACTONE (10%), SALICYLIC ACID (2%), TROMETHAMINE (5%), Preservative (1.1%), Niacinamide (2%), Propanediol (4.95%), Oleanolic Acid (0.05%)
Oil phase 15 wt % or less

### Example 4

Additionally, a composition of Example 4 having the following entire ingredients was prepared. As a result of observation, the formulation was well made, and had excellent formulation stability and excellent water-bursting feel.
Water phase (80 wt %): Purified water (53.9%), Sodium chloride (1%), GLUCONOLACTONE (10%), SALICYLIC ACID (2%), TROMETHAMINE (5%), Preservative (1.1%), Niacinamide (2%), Propanediol (4.95%), Oleanolic Acid (0.05%)
Oil phase (20 wt %): DICAPRYLYL CARBONATE 12.4925%, SYNTHETIC WAX 7%, CAPRYLYL DIMETHICONE ETHOXY GLUCOSIDE 0.0075%, LAURYL POLYGLYCERYL-3-POLYDIMETHYLSILOXYETHYL DIMETHICONE 0.5%

### Comparative Example 4

Additionally, a composition of Comparative Example 4 was prepared in the same method as that of Example 1, except that DIMETHICONE/PEG-10/15 CROSSPOLYMER and DIMETHICONE, which are a PEG-based surfactant and silicone oil, are contained instead of the silicone emulsifier containing the ethoxy glucoside derivative. As a result of observation, the formulation was well made, and had excellent formulation stability and excellent water-bursting feel.
Water phase (85 wt %): Purified water (58.9%), Sodium chloride (1%), GLUCONOLACTONE (10%), SALICYLIC ACID (2%), TROMETHAMINE (5%), Preservative (1.1%), Niacinamide (2%), Propanediol (4.95%), Oleanolic Acid (0.05%)
Oil phase (15 wt %): DIMETHICONE 7.485%, SYNTHETIC WAX 7%, DIMETHICONE/PEG-10/15 CROSSPOLYMER 0.415%, LAURYL POLYGLYCERYL-3-POLYDIMETHYLSILOXYETHYL DIMETHICONE 0.1%

What is claimed is:

1. A water-in-oil type solid stick composition containing a large amount of water phase, wherein the solid stick composition comprises an oil phase and a water phase,
wherein the oil phase comprises a silicone emulsifier containing an ethoxy glucoside derivative,
the water phase comprises an acidic ingredient, and
the water phase is 35 wt % or more based on the total weight of the solid stick composition.

2. The water-in-oil type solid stick composition containing a large amount of water phase according to claim 1, wherein a pH of the composition is 3 to 6.

3. The water-in-oil type solid stick composition containing a large amount of water phase according to claim 1, wherein the silicone emulsifier containing the ethoxy glucoside derivative is present in an amount of 0.001 to 10 wt % based on the total weight of the composition.

4. The water-in-oil type solid stick composition containing a large amount of water phase according to claim 1, wherein the silicone emulsifier containing the ethoxy glucoside derivative comprises at least one selected from caprylyl dimethicone ethoxy glucoside and dimethicone ethoxy glucoside.

5. The water-in-oil type solid stick composition containing a large amount of water phase according to claim 1, wherein the oil phase further comprises at least one selected from the group consisting of hydrocarbon oil, a wax, and an additional emulsifier.

6. The water-in-oil type solid stick composition containing a large amount of water phase according to claim 5, wherein the carbon number of the hydrocarbon oil is 20 or less.

7. The water-in-oil type solid stick composition containing a large amount of water phase according to claim 5, wherein the hydrocarbon oil comprises at least one selected from the group consisting of isododecane and dicaprylyl carbonate.

8. The water-in-oil type solid stick composition containing a large amount of water phase according to claim 5, wherein the hydrocarbon oil is present in an amount of 5 to 12 wt % based on the total weight of the composition.

9. The water-in-oil type solid stick composition containing a large amount of water phase according to claim 5, wherein a melting point of the wax is 50° C. or more.

10. The water-in-oil type solid stick composition containing a large amount of water phase according to claim 5, wherein the wax is present in an amount of 5 to 10 wt % based on the total weight of the composition.

11. The water-in-oil type solid stick composition containing a large amount of water phase according to claim 5, wherein the additional emulsifier comprises at least one of a silicone-based emulsifier and a polyglyceryl-based non-silicone-based emulsifier.

12. The water-in-oil type solid stick composition containing a large amount of water phase according to claim 11, wherein the silicone-based emulsifier comprises at least one of lauryl polyglyceryl-3-polydimethylsiloxyethyl dimethicone and polyglyceryl-3-polydimethylsiloxyethyl dimethicone.

13. The water-in-oil type solid stick composition containing a large amount of water phase according to claim 11, wherein the polyglyceryl-based non-silicone-based emulsifier comprises at least one selected from the group consisting of polyglyceryl-5-polyricinoleate, polyglyceryl-4-isostearate, polyglyceryl-3-polyricinoleate, polyglyceryl-2-dipolyhydroxystearate, diisostearoyl polyglyceryl-3-dimer dilinoleate, polyglyceryl-4-diisostearate/polyhydroxystearate/sebacate, polyglyceryl-2-isostearate, polyglyceryl-3-diisostearate, and polyglyceryl-2-isostearate/dimer dilinoleate copolymer.

14. The water-in-oil type solid stick composition containing a large amount of water phase according to claim 5, wherein the additional emulsifier is present in an amount of 0.1 to 5 wt % based on the total weight of the composition.

15. The water-in-oil type solid stick composition containing a large amount of water phase according to claim 1, wherein the acidic ingredient comprises at least one selected from the group consisting of a polyhydroxy acid (PHA), a beta hydroxy acid (BHA), and an alpha hydroxy acid (AHA).

16. The water-in-oil type solid stick composition containing a large amount of water phase according to claim 1, wherein the acidic ingredient comprises at least one selected from the group consisting of gluconolactone, salicylic acid, glycolic acid, lactic acid, citric acid, ascorbic acid, ethyl ascorbic acid, succinic acid, and ferulic acid.

17. The water-in-oil type solid stick composition containing a large amount of water phase according to claim 1, wherein the acidic ingredient is present in an amount of 1 to 15 wt % based on the total weight of the composition.

18. The water-in-oil type solid stick composition containing a large amount of water phase according to claim 1, wherein the composition does not comprise a PEG-based surfactant and silicone oil.

19. The water-in-oil type solid stick composition containing a large amount of water phase according to claim 1, wherein the composition is a cosmetic composition.
